(19) United States
(12) Patent Application Publication
(10) Pub. No.: US 2013/0103100 A1
(43) Pub. Date: Apr. 25, 2013

(54) FIXATION IMPLANT

(71) Applicant: SYNERGY BIOSURGICAL AG, Zug (CH)

(72) Inventor: Kurt RUFFIEUX, Thalwill (CH)

(73) Assignee: SYNERGY BIOSURGICAL AG, Zug (CH)

(21) Appl. No.: 13/652,095

(22) Filed: Oct. 15, 2012

Related U.S. Application Data
(63) Continuation of application No. PCT/CH2011/000075, filed on Apr. 11, 2011.

(30) Foreign Application Priority Data
Apr. 13, 2010 (CH) 529/10

(51) Int. Cl. A61B 17/86

(52) U.S. Cl. CPC A61B 17/8645
USPC 606/321

(57) ABSTRACT

A fixation implant tightly secures ligament transplants and the like on or in bone tissue. The fixation implant has an elongate body, which has a front end for application and includes at least in some areas an expandable material which, after the implantation, exerts an expansion pressure on a surrounding bone substance. The body has an outer wall, which is provided at least in some areas with profiles extending substantially transverse to the longitudinal extent. The body is provided at its rear end with an engagement means for an insertion instrument, or it has an opening into a central recess for an expansion body. The expandable material is arranged substantially along the entire longitudinal extent of the body. The expandable material can exert pressure on the profiled areas in such a way that the external diameter of the body increases.

### RELATED APPLICATIONS

This application claims priority as a continuation application under 35 U.S.C. §120 to PCT/CH2011/000075, which was filed as an International Application on Apr. 11, 2011 designating the U.S., and which claims priority to Swiss Application No. 00529/10 filed in Switzerland on Apr. 13, 2010. The entire contents of these applications are hereby incorporated by reference in their entireties.

### FIELD

Disclosed is a fixation implant for tightly securing ligament transplants and the like in or on bones.

### BACKGROUND INFORMATION

In the event of a fracture of the cruciate ligament, in many cases a ligament transplant is used, whose end is attached in one hole pre-drilled into the femur and one pre-drilled into the tibia. The attachment of the ligament transplant is carried out in many cases by means of so-called interference screws. Interference screws can include, for example, conically tapering bone screws of different external diameters that are provided with external threading. With such interference screws, the ligament transplant, in most cases a tendon, is fixed in a pre-drilled bone bed. The fixing of the ligament transplant is done in this case by clamping between the wall of the hole in the bone and the interference screw. Interference screws can be cannulated, so that when screwing into the bone, torque can be transferred over the entire length of the screw. As a result, excessive screw-in resistance causing the interference screw to break can be prevented. In addition to metal interference screws, interference screws made from biocompatible plastics can be used, which can also be resorbable.

In the fixation of ligament transplants to eliminate cruciate ligament fractures, the transplanted tendon can be injured by excessively strong squeezing. The transplanted tendon can have the property of mechanically relaxing, by which the clamping force of the interference screw or an alternative clamp fastening, for example a plate and screws, can be reduced, and as a result, the tendon can slide out of the bone bed again. Tendons have a slippery consistency. To fix this slippery tendon in the bone bed, interference screws and such fixation elements can be equipped with retaining ribs. In the case of the interference screws, the external threading can perform this function. In the clamping in the bone bed, great force is exerted on the tendon, which can be concentrated on the threaded edges or the edges of the retaining ribs. As a result, it can lead to damage of the tendon. To counteract such damage, interference screws with rounded threaded edges can be used. Such screws can have the drawback that they offer inadequate retention relative to the tendon and can form an inadequate positive fit on the boundary wall of the bone hole.

A tendon is softer than a bone. However, a tendon can offer specific compression relative to an interference screw that is screwed into a bone bed, a greater resistance than the bone itself. This can result in the surgeon exerting excessive screw-in torque when screwing in the interference screw in order to overcome this elevated resistance. As a result, in addition to the tendon, the surrounding bone may also be damaged. The tendon is a viscoelastic tissue, to which the volume can adapt when maintaining pressure over time. This can result in a volume reduction of the transplant to be fixed with the result that the clamping force of the fixation element that is used is reduced, and it can result in a loosening between the fixation element and tendon. Because of the above-described problem, a surgeon may be inclined to screw a considerably larger interference screw into the bone canal than that which corresponds to the diameter with which the bone canal was drilled, with this being done even though one end of the tendon projects into the bone canal. This measure may be used, for example, in older patients whose bone substance no longer offers sufficient hold. Owing to the massive displacement of bone and tendon, the interference screw with the larger diameter can then cause the bone and tendon to be displaced in or from the drilled hole at one point, which no longer corresponds to the anatomically desired insertion point. For example, only in the case of successful healing in such cases can an unnecessarily large hole remain in the bone, which in the case of resorbable screw material often heals only with scar tissue and not with bone substance. The combination of tendon damage, tendon volume reduction, bone expansion and still weaker fixation can result in a loosening between tendon and interference screw or the same fixation elements and in an unreliable transplant fixation. This can ultimately result in a failure of the treatment.

In U.S. Pat. No. 5,084,050, a fixation element in the form of a bone pin is described. Over time, upon contact with bodily fluids, the bone pin that is designed as a hollow body can exhibit certain swelling properties after its implantation, by which its outer diameter increases. The bone pin is used to attach metal bone screws in the bone tissue. Accordingly, a threaded structure is provided in the interior of the bone pin. The outer contour of the bone pin is provided with rounded ribs or a structure that consists of spherical surfaces to protect the surrounding bone substance as much as possible. For a fixing of ligament transplants between the outer wall of the bone pin and the bone substance, the fixation element that is described in U.S. Pat. No. 5,084,050 is hardly suitable, since the latter is only inadequately clamped because of the rounded outer contour of the bone pin. As another possible application of the fixation element, the fixing of ligament-bone cylinders inside the bone pin with subsequent ingrowth of the bone is described in U.S. Pat. No. 5,084,050. Upon swelling of the hollow bone pin that is optionally closed on one side, it is primarily the central receptacle hole for the metal screw or the bone cylinder that is closed. In this case, however, little or no outward pressure is produced.

In U.S. Patent Application Publication No. 2008/0167717, a combination that consists of a bone-fixing plate and bone screws is described. The bone screw can have an area that can swell upon contact with bodily fluids to achieve a tight connection between the bone-fixing plate and the bone screw by enlarging the outer diameter of this area. For example, a clamp fixing of a ligament transplant in a bone bed between the outside of the bone screw and the wall of a hole in the bone is not mentioned in this document.

Various exemplary embodiments of so-called suture anchors are described in U.S. Pat. No. 6,152,949. Suture anchors are cylindrical fixation elements that are anchored in bone or soft tissue to fix muscle tissue with a thread that is attached to the anchor. The thread can be run through an axial hole in the suture anchor. The suture anchors described there can also be produced from a material that swells upon contact with bodily fluids. For example, suture anchors are not suitable for a clamp fixation of ligament transplants in bone beds.

### SUMMARY

According to an exemplary aspect, disclosed is a fixation implant for tightly securing a ligament transplant on or in a bone, the fixation implant comprising: an elongated body that includes a front end, and at least partially contains an expandable material that, after implantation, exerts an expansion pressure on a surrounding bone substance, wherein the elongated body includes an outer wall which includes, at some areas, profiles that extend substantially transverse to a longitudinal direction, wherein the elongated body on its rear end has engagement means for an insertion instrument, or an opening in a central receptacle for an expansion body, wherein the expandable material is arranged substantially along the entire longitudinal extent of the elongated body, and wherein the expandable material is arranged such that, upon expansion, the expandable material exerts pressure on the areas that includes the profiles in such a way that the external diameter of the body increases.

According to an exemplary aspect, disclosed is a method of tightly securing a ligament transplant in or on a bone, the method comprising: implanting the fixation implant according to an exemplary aspect into a surrounding bone substance.

### BRIEF DESCRIPTION OF THE DRAWINGS

Other exemplary embodiments of the disclosure follow from the description below with reference to the drawings. Any diagrammatic representations in the drawings are not necessarily true to scale.

FIG. 1 shows a first embodiment of a fixation implant in the form of an interference screw, in accordance with an exemplary aspect;

FIGS. 2a and 2b show two views of a second embodiment that is modified relative to FIG. 1, in accordance with an exemplary aspect;

FIGS. 3a and 3b show two views of a third embodiment in the form of an interference screw, in accordance with an exemplary aspect;

FIGS. 4a and 4b show two views of a fourth embodiment in the form of an interference screw, in accordance with an exemplary aspect;

FIG. 5 shows an exemplary embodiment of a fixation implant that is modified relative to the embodiment according to FIG. 4, in accordance with an exemplary aspect;

FIG. 6 shows another embodiment of a fixation implant, designed as an interference screw, in the implanted state, in accordance with an exemplary aspect;

FIG. 7 shows an embodiment of a fixation implant with shell segments and wedge, in accordance with an exemplary aspect;

FIG. 8 shows another embodiment of the fixation implant that is modified relative to the embodiment according to FIG. 7, in accordance with an exemplary aspect;

FIGS. 9a and 9b show another embodiment of a fixation implant that is designed for the fixing of several ligament transplants, in accordance with an exemplary aspect;

FIGS. 10a and 10b show another embodiment of a fixation implant, in accordance with an exemplary aspect;

FIGS. 11-15 show cross-sections of other exemplary embodiments of a fixation implant, in accordance with exemplary aspects;

FIGS. 16 and 17 show cross-sections of two other exemplary embodiments of a fixation implant, in accordance with an exemplary aspect;

FIG. 18 shows a view on the rear end of another exemplary embodiment of a fixation implant, in accordance with an exemplary aspect; and

FIG. 19 shows a perspective view of the fixation implant according to FIG. 18, in accordance with an exemplary aspect.

### DETAILED DESCRIPTION

According to an exemplary aspect, provided is a fixation element for fastening ligament transplants and the like to or in bones, which initially fixes the transplant and maintains the clamping pressure on the ligament transplant despite the volume shrinkage of the transplant.

According to an exemplary aspect, a fixation implant for tightly securing ligament transplants and the like on or in bones is provided that has exemplary features.

An exemplary fixation implant can be used, for example, to tightly secure ligament transplants and the like on or in bone tissue. It can have an elongated body that has a front end for application and at least in places includes an expandable material that exerts an expansion pressure on a surrounding bone substance after implantation. The body can have an outer wall, which is equipped at least in places with profiles that run substantially crosswise to the longitudinal extension. On its rear end, the body can be equipped with an engagement means for an insertion instrument, or it can have an opening in a central receptacle for an expansion body. The expandable material can be arranged substantially over the entire longitudinal extension of the body. The areas that are provided with profiles can be exposed to pressure by the expandable material in such a way that the external diameter of the body increases.

For example, by the fixation implant having an expandable material, which can be arranged substantially over the entire longitudinal extent of its body, as uniform a pressure as possible can be exerted on the ligament transplant and on the bone structure surrounding the fixation implant. For example, the pressure develops ab initio, i.e., directly after the insertion of the fixation implant, and remains, even if a widening of the receptacle hole in the bone takes place. The fixation implant can thus have a self-readjusting function.

For example, the expansion pressure and the readjusting function are ensured by the expandable material, which can comprise, for example, an elastically compressible biocompatible plastic. In an exemplary embodiment, the expandable material comprises a material that can swell upon contact with bodily fluids. By the fixation implant already exerting pressure on the ligament transplant and the surrounding bone structure immediately after the introduction into the receptacle hole in the bone and because of its self-readjusting function, the external diameter of the fixation implant can be kept comparatively small. As a result, only a relatively small receptacle hole has to be made in the bone, which can have an advantageous effect on the healing process. For example, the external diameter of the fixation implant can be approximately 5 mm to 10 mm. By an open geometry of the fixation implant that is as thin as possible, the amount of the material that is used can be reduced. At the same time, the ingrowth of the ligament transplant may be desired, since bone and scar tissue can grow through the gaps.

By the expansion pressure that develops ab initio, as large a contact surface as possible, which promotes a large-surface ingrowth, can be achieved between the bone and the ligament transplant. The fixation implant can also be arranged at the free end of the ligament transplant (e.g., of a tendon) in its interior. Then, the thus charged transplant can be pressed into the receptacle hole in the bone. By the expansion pressure and the self-adjusting effect of the fixation implant, the ligament transplant can be pressed on all sides onto the bone wall, by which a large contact surface that promotes ingrowth can be achieved.

In an exemplary embodiment, the readjustment of the fixation implant in which the material that is used swells is achieved. For example, resorbable or non-resorbable polymers can be used. For example, an addition of calcium phosphates to high-molecular polylactides (e.g., 200,000 gmol) can result in a constant volume increase for 40 weeks. The calcium phosphate can lead to an osmotic difference that results in absorption of water. The thus produced pressure in the fixation implant can result in its swelling. For example, when using 10 mol % tricalcium phosphate, the volume in an in vitro test at 37° C. increases by 3% after 2 weeks, by 13% after 10 weeks, and by almost 25% after 24 weeks. In vivo studies show that when adding only 1% sodium phosphate, for example, a volume increase of 13% can be achieved after 2 weeks, 30% after 6 weeks, and around 100% after 28 weeks. Accelerated in-vitro studies at an elevated temperature of 55° C. show, for example, that the addition of 10 mol % sodium phosphate as early as after 10 days results in a volume increase of 100%, while the same polylactide without additives undergoes a volume increase of only 4% in the same period.

A counterpressure, e.g., bone tissue, can result in a smaller degree of swelling. Thus, the fixation implant can already have a self-readjusting effect starting at an early point in time and swelling. The expandable material can comprise a resorbable polymer with a molecular weight of <100,000 g/mol. For example, when the value drops below this molecular weight limit, the strength of the polymer can decrease significantly more quickly. This can be substantiated with an increased mobility of the molecule. For example, polylactides with an L/D ratio of approximately 70/30 can increase significantly in volume starting from about 20 weeks. In the case of polylactides from racemic D, L-lactide, for example, the volume increase can be carried out as early as after 10 weeks. This can correspond approximately to the point in time when the value drops below a molecular weight of approximately 100,000 g/mol.

In another exemplary embodiment, the fixation implant can include several layers of expandable materials, which, on the one hand, make possible the initial fixation, and, on the other hand, make possible a subsequent upholding of the pressure by self-adjustment. This can be carried out by, for example, the introduction of pores into the fixation implant, via which the liquid absorption and thus swelling behavior can be controlled.

The biocompatible expandable material can also be produced from non-resorbable materials. For example, it can be a non-resorbing hydrogel or a salt-filled cushion, which takes up liquid and swells by osmotic effects. Combinations of expandable materials and non-expandable materials are also possible. Thus, in an additional exemplary embodiment, the outside part of the fixation implant can include comparatively hard shells with a slip-proof outer contour, while the core area of the fixation implant is produced from an expandable, relatively soft material. Textile materials can also be used.

In an exemplary embodiment, the expandable material of the fixation implant mechanically is not heavily pressure-loaded, for example, in connection with silicone or other soft components. Exemplary embodiments therefore provide for the expandable material to be arranged in the fixation implant in such a way that it can optimally exert its pressure. For example, this can be a zone, extending over the length of the fixation implant, in its interior. To control a swelling behavior and the swelling speed, a swellable material that is used can be arranged in such a way that, for example, bodily fluid cannot force its way outward, but can penetrate into the interior. In an exemplary embodiment, an outer shell that encloses the swellable material is produced from a porous material, for example, calcium phosphate, PEEK, polylactide, polyglycolide or the like. Porosity can also be achieved by smaller bores, holes or capillaries. For example, bodily fluid can get into the interior to form swellable material, while the latter itself, however, remains collected within the shell. In another exemplary embodiment, the swellable material is surrounded by a mechanically resistant, liquid-permeable biocompatible membrane.

For example, to mitigate or prevent too much of the expandable material from going outward over time, the expandable material can be designed with varying expansion behavior or for setting a gradient with respect to the expansion. For example, when using pairs of salt in silicone or salt in polyurethane deep within the swellable material, a larger proportion of salt (e.g., 40-80 percent by volume) can be provided, while the proportion of salt on the surface of the expandable material can be relatively small (e.g., 0-10 percent by volume). As a result, the interior of the swellable material can expand considerably more, and an overall more homogeneous distribution of force can be achieved. At the same time, the expandable material can thus be held on site.

In an exemplary embodiment, the fixation implant is designed according to a type of an interference screw with an external diameter of approximately 5 mm to 10 mm. Unlike a comparative interference screw, such exemplary interference screw can, for example, have a solid body and is not cannulated. On its rear end, a cover plate that includes a rigid, non-expandable material can be molded-on, in which an engagement means with torque transfer surfaces can be formed. For example, the engagement means cab be a Torxx or cross-slot receptacle, whose depth is less than ¼ of the axial length of the fixation implant. For example, the fixation implant, which can have the form of an interference screw, can include or entirely consist of, for example, poly-D,L-lactide with an L-lactide to D-lactide ratio of 85/15. This material can have a molecular weight of <100,000 g/mol and can have a proportion of approximately 10% (w/w) sodium phosphate. For example, immediately after the introduction into the receptacle hole in the bone, the implant begins to take up bodily fluid, and the swelling process begins. The counterpressure of the fixation transplant and the surrounding bone can counteract an increase in volume. For example, if the fixation implant buckles, the counterpressure decreases, and the fixation implant further swells. Over the course of 1-2 years, the fixation implant can decay completely. By then, for example, the ingrowth process of the ligament transplant that is first held by clamping will be long since completed.

For example, an alternative variant of the fixation implant includes two outer-ribbed half-shells that are pressed outward by an inside wedge. A ligament transplant can be fixed by clamping between the fixation implant and the bone wall. The half-shells can include or consist of pure poly-D,L-lactide with a ratio of 70% L-lactide and 30% D-lactide with a molecular weight of approximately 200,000 g/mol. The wedge can include or consists of polylactide-co-glycolide with a molecular weight of <100,000 g/mol, which is mixed with >50% (w/w) tricalcium phosphate (TCP). The high content of TCP can allow a quick diffusion of bodily fluid in the wedge and thus a quick swelling of the same. This can lead to a pushing apart of the half-shells and thus to a self-readjusting effect of the fixation implant. The wedge can degrade within approximately 6-9 months, and the half-shells within approximately 2 years.

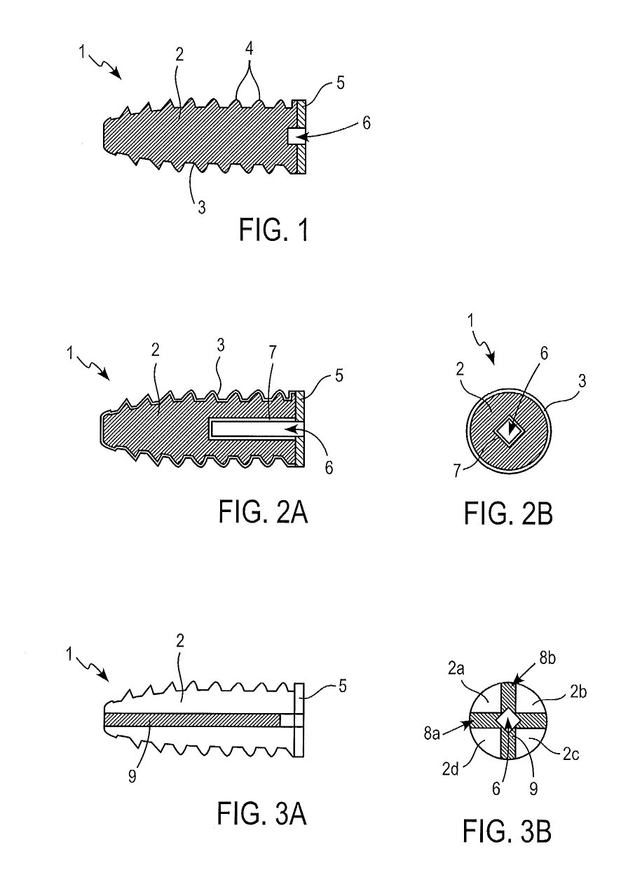

In an exemplary embodiment, FIG. 1 diagrammatically shows a fixation implant that is referred to with the overall reference number 1 and that has the external form of an interference screw. The fixation implant 1 has a solid body 2 that can include a material that can expand, for example, swell, upon contact with bodily fluids. The outer wall 3 of the approximately torpedo-like body 2 can be equipped with thread-like profiles 4. On its rear end, a cover plate 5 that includes a non-expandable material can be arranged, which is equipped with a receptacle 6, used as an engagement means with torque transfer surfaces, for an insertion instrument, for example a screw bit or the like. The cover plate 5 can reduce the absorption of bodily fluid on the rear of the implant, so that the implant expands as little as possible in an axial direction and, for example, cannot escape from the receptacle hole in the bone. The swellable material can be provided, for example, with salt crystals. To control the swelling behavior, the salt content in a central area of the swellable body 2 can be higher than in the areas that are near the surface. The fastening of a ligament transplant, for example, a piece of tendon, in a bone bed can be carried out by clamping between the outer wall 3 of the body 2 of the fixation implant that is inserted into a bone hole and the surrounding bone substance.

In an exemplary embodiment, an exemplary fixation implant shown in FIG. 2 has an interference screw design that is similar to the exemplary embodiment according to FIG. 1. In FIG. 2, the fixation implant that is given the overall reference number 1 has a body 2 that includes an expandable material. On its rear end, a cover plate 5 is equipped with a receptacle 6 for torque-transferring insertion instruments. The receptacle 6 projects into the body 2 of the fixation element 1 and has a coating layer 7 that includes a non-expandable material, which offers resistance inward upon contact with material of the body 2 that swells with bodily fluid. The outer wall 3 of the body 2 that is equipped with thread-like profiles can include a non-expandable material, which provides for a harder surface of the fixation implant 1. As a result, the positive hold relative to the fixation implant and the surrounding bone material can be improved. The outer wall 3 that includes non-expandable material can allow the penetration of bodily fluids into the swellable material of the body 2 and can buckle under the inner swelling pressure, so that the fixation implant 1 can increase its external diameter. To control the swelling behavior and to facilitate the expansion of the swellable material, canals, slots or scores can also be arranged in the outer wall 3 (not shown).

In an exemplary embodiment, FIGS. 3a and 3b show two diagrammatic views of an exemplary fixation implant 1 that is designed as an interference screw. The body 2 of the fixation implant includes four shell segments 2a, 2b, 2c, and 2d. The outer walls of the shell segments 2a-2d are equipped with profiles which are supplemented in a thread-like manner over the periphery of the fixation implant 1. The shell segments 2a-2d are separated from one another by slots 8a, 8b, for example, that can run perpendicularly to each other. In the slots 8a, 8b, a material 9 that can swell upon contact with bodily fluids can be arranged. On its rear end, the fixation implant 1, as shown, can be connected to a cover plate 5, in which a receptacle 6 for a torque-transferring insertion instrument is arranged.

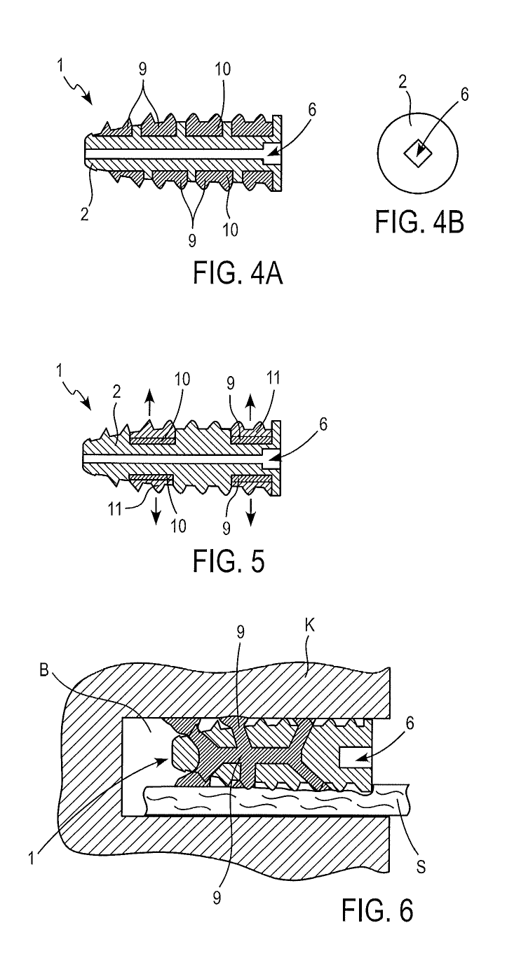

In an exemplary embodiment, an exemplary fixation implant that is depicted in FIGS. 4a and 4b is referred to with the overall reference number 1. Its external shape to a large extent corresponds to that of a torpedo-shaped interference screw. The body 2 of the fixation implant 1 includes a central structural skeleton that includes a non-expandable material. In addition, the body 2 has several receptacles 10 that run in the peripheral direction and that are made open toward the periphery. In the receptacles 10, in each case a material 9 that can swell upon contact with bodily fluids is arranged. In an alternative exemplary embodiment of the fixation element, not shown, the receptacles can also run, for example, in an axial direction. On its rear end, the fixation implant has an engagement means in the form of a receptacle 6 for a torque-transferring insertion instrument. The receptacle 6 can run through the entire body 2 of the fixation implant 1.

In an exemplary embodiment, an exemplary fixation implant 1 that is depicted in FIG. 5 corresponds to a large extent to that of FIGS. 4a and 4b. Unlike the fixation element depicted there, the expandable material that is arranged in the receptacles 10 in the peripheral direction or axially is covered by a non-expandable, harder coating layer 11 toward the periphery. For example, the coating layer 11 can be designed in a circle or spiral. The coating layer 11 can be designed in such a way that it can be displaced by the swelling pressure of the expandable material 9 and can be pressed against the surrounding bone structure. The outer surface of the coating layer 11 can have ribs or edges that provide for a better hold of the ligament transplant, for example a tendon, in the bone. To insert the implant, the expandable material can be compressed and thus can ensure an expansion pressure with an ab initio effect on the ligament transplant after the insertion into the receptacle hole.

In an exemplary embodiment, FIG. 6 shows an exemplary fixation implant 1, which is designed similar to the fixation implant that is depicted in FIGS. 4a and 4b. The body 2 of the fixation implant 1 in turn is formed by a central structural skeleton. The swellable material 9 that is in contact with bodily fluids is arranged in the body 2 in receptacles designed like canals. At its rear end, the body has a receptacle for an insertion instrument. The fixation implant 1 that is designed as an interference screw is inserted into a hole B in a bone K and fixes a ligament transplant, for example a tendon S, by clamping on the bone canal. In such exemplary embodiment, the fixing is not carried out by a mechanical change, for example, overall diameter increase of the fixation implant 1, but rather the swelling material exits from one or more openings of the body 2. In this way, the tendon S can be fixed, and also the interference screw in the bone canal B can be stabilized.

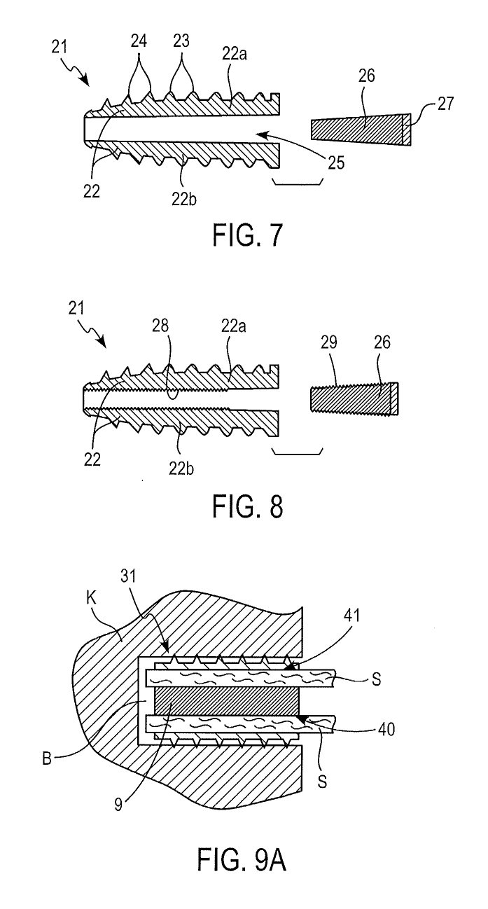

In an exemplary embodiment, an exemplary fixation implant that is diagrammatically depicted in FIG. 7 bears the overall reference number 21. Its body 22 comprises two or more shell segments 22a, 22b that are separated from one another by longitudinal slots and are fixed to hold to one another in a way that is not shown in more detail but can be separated from one another by an expansion pressure. The shell segments 22a, 22b include a material, for example, that itself is not expandable. On the outer walls 23, the shell segments have profiles 24, which can provide for a better hold of a ligament transplant and for a better anchoring of the fixation transplant in a bone hole. The shell segments can have openings that make possible the access of bodily fluid inside the fixation implant. The expansion pressure that allows for an anchoring can be applied by an expansion cone 26, which can be driven into a central hole 25 of the body 22 that is limited by the shell segments 22a, 22b. The central hole can be designed conically tapered in the direction of insertion. The expansion cone 26 can partially or completely consist of a material that can expand upon contact with bodily fluids. On its rear end, the expansion cone 26 can be equipped with a cover plate 27, which can reduce or prevent bodily fluids from entering from the rear and can offer resistance to an expansion of the expansion cone from the body 2.

In an exemplary embodiment, an exemplary fixation implant 1 that is depicted in FIG. 8 and provided with the reference number 21 corresponds to a large extent to the fixation implant of FIG. 7. Additional ribs 28 or the like are applied on the inner wall of the body 22, and said ribs 28 work together in a positive manner with correspondingly designed structures 29 on the expansion cone 26 and keep the expansion cone 26 from sliding back.

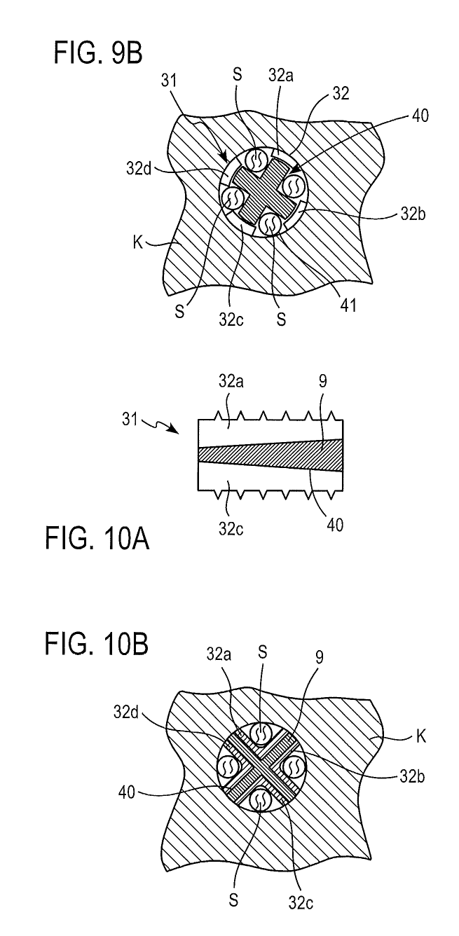

In an exemplary embodiment, an exemplary fixation implant that is depicted in FIGS. 9a and 9b bears the overall reference number 31. Its body 32 has several shell segments 32a-32d, which are separated from one another by longitudinal slots 40. In the shell segments 32a-32d, axially running recesses 41 are arranged, which, for example, can taper conically from an insert opening on the rear end of each shell segment 32a-32d in the direction of the front end of the body 32. The recesses 41 can be designed open toward the periphery of the body 32. Each conical recess can be used to accommodate a ligament transplant, for example a tendon S. The exemplary embodiment of the fixation implant 31 can therefore accommodate and hold up to four tendons S. Expandable, for example, swellable, material 9 is arranged in the longitudinal slots 40.

For example, for insertion, the expandable material can be compressed, and the shell segments 32a-32d can be pressed apart after the introduction. As a result, the tendon material S can be pressed against the wall of the bone canal B. If the bone canal B is further widened, the material 9 that can swell upon contact with bodily fluids cab ensure that the clamping pressure is maintained on the tendons S. The outer surface of the shell segments adjacent to the tendon material can have an elevated roughness or be equipped with ribs, mandrels or edges to keep the tendon material from sliding. The sections facing the bone canal B, shell segments 32a-32d, can have additional anchoring aids such as edges, spikes or mandrels, so that they find a better hold in the bone canal B.

In an exemplary embodiment, an exemplary fixation implant depicted in FIGS. 10a and 10b bears the overall reference number 31. It corresponds to a large extent to the exemplary embodiment according to FIGS. 9a and 9b. In FIGS. 10a and 10b, the shell segments 32a-32d that are separated from one another by longitudinal slots 40 can be widened by a central swelling body 9, whose shape is specifically laid out on the inner contour of the shell segments 32a-32d. In an exemplary embodiment, the central swelling body 9 has, for example, a cross-shaped cross-section. A central screw can also be provided that includes a material that can swell upon contact with bodily fluids and changes corresponding to its external diameter. In the longitudinal direction, the central swelling body 9 has a wedge-like configuration.

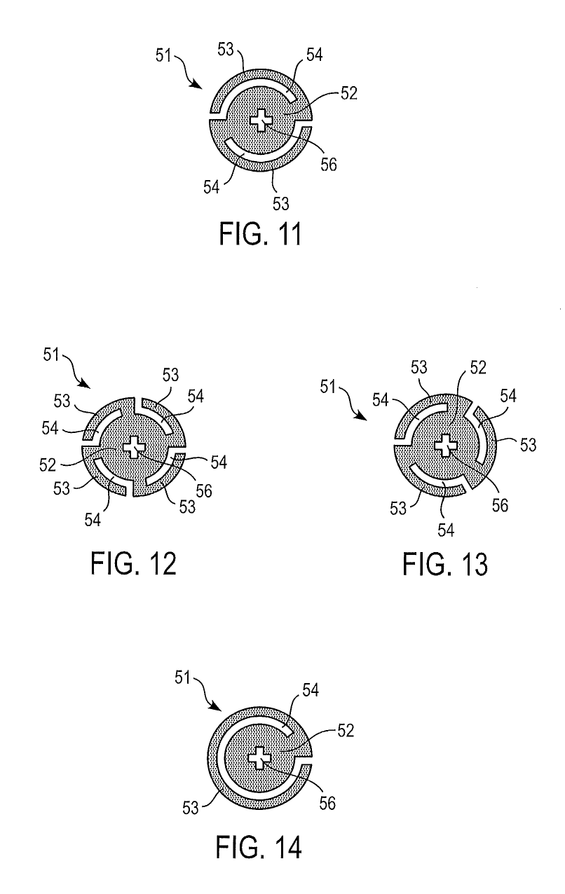

In exemplary embodiments, FIGS. 11-15 show cross-sections of additional exemplary configurations for fixation implants according to exemplary aspects, which in each case are given the overall reference number 51. A common feature of the exemplary embodiments is a body 52 that comprises a structural skeleton, shown in gray in the figures, including a non-expandable material, which is provided with radially expandable areas 53, which radially adjoin at least areas 54 that are filled in places with expandable material, areas that are indicated in white in the figures. The expandable material can be an elastically compressible material and/or a material that can swell upon contact with bodily fluids. The non-expandable carrier material of the body 52 includes, for example, polylactide (PLA). The radially expandable areas 53 are designed like shell segments and are coupled axially on one side at the periphery of the body 52. Each of the exemplary embodiments has a central receptacle 56 on its rear end for an insertion instrument. The receptacle 56 can extend from the rear end area into the interior of the body 52. On the outer wall of the body 52, profiles that are not depicted in more detail, such as, e.g., threads, edges, spikes, mandrels, etc., can be designed, so that a ligament transplant can be better held, and the fixation implant in the bone canal finds a better anchoring.

In an exemplary embodiment, FIG. 11 shows an exemplary fixation implant 51 with two wing-like shell segments 53 coupled on one side on the periphery. The shell segments 53 adjoin slots 54 running in the peripheral direction and provided in the body 52, slots that extend axially through the body 52. Because of an expansion pressure that is exerted by expanding material arranged in the slot 54, the wing-like shell segments 53 are pressed radially outward.

In an exemplary embodiment, the exemplary fixation implant 51 depicted in FIG. 12 corresponds to a large extent to the exemplary embodiment according to FIG. 11. In FIG. 12, four wing-like shell segments 53 are coupled to the central body 52, which are pressed radially outward by the expanding material that is arranged in the slots 54.

In an exemplary embodiment, FIG. 13 shows an exemplary fixation implant 51 with three wing-like shell segments 53, while in FIG. 14, an exemplary fixation implant 51 is depicted with only one wing-like shell segment 53, which extends almost in an arc over the entire periphery of the body 52 and adjoins an arc-shaped slot 54, which is filled with the expandable material.

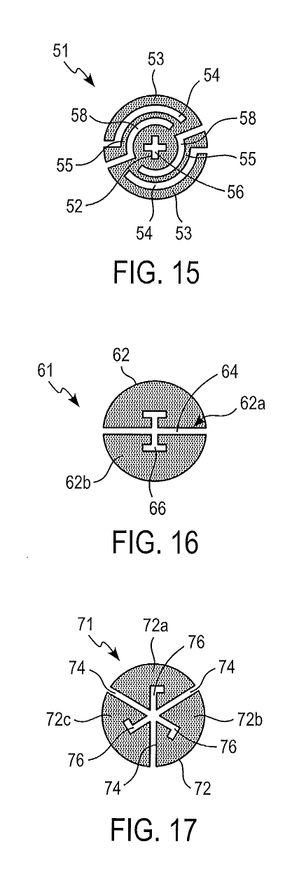

In an exemplary embodiment, FIG. 15 shows an exemplary embodiment of a fixation implant 51 that has two series of concentrically arranged shell segments 53, 55, which in each case are separated from one another by arc-shaped slots 54, 58 and from the supporting structural skeleton of the body 52. An expandable material is arranged in the slots 54, 58. A receptacle that is arranged in the body 52 for an insertion instrument bears the reference number 56.

In an exemplary embodiment, an exemplary fixation implant that is depicted in FIG. 16 is given the overall reference number 61. It has a body 62, which is composed of two half-shells 62a, 62b, which are connected to one another in a way that is not depicted in more detail. The half-shells 62a, 62b include a non-expandable material, for example polylactide (PLA), and are separated from one another by a longitudinal slot 64 that runs axially and that is filled with an expandable material. The expandable material can be an elastically compressible material or a material that can swell upon contact with bodily fluids. Because of the expansion pressure of the expandable material, the two half-shells 62a, 62b can be pressed apart, whereby the external diameter of the fixation implant 61 is increased to the desired extent. The receptacle 66, recessed in the rear area of the body 62, for an insertion instrument can have the shape of, for example, an I, according to an exemplary embodiment.

In an exemplary embodiment, an exemplary fixation implant depicted in cross-section in FIG. 17 is provided with the reference number 71. It has a body 72 that includes a non-expandable material, for example PLA, which is composed of three shell segments 72a, 72b, 72c, which are connected to one another in a way that is not depicted in more detail. The shell segments 72a, 72b, 72c are separated from one another by three longitudinal slots 74 that run radially and that are filled with expandable material. The expansion pressure of the expanding material presses the three shell segments 72a, 72b, 72c apart radially with enlargement of the external diameter. On the rear end area, the body 72 has a receptacle 76 for an insertion instrument, which, for example, has the shape of a three-armed star with vanes running at right angles thereto. The three arms of the star-shaped receptacle 76 are arranged offset relative to the longitudinal slots 74 by approximately 60°, so that they pass approximately through the centers of the respective shell segment 72a, 72b, 72c.

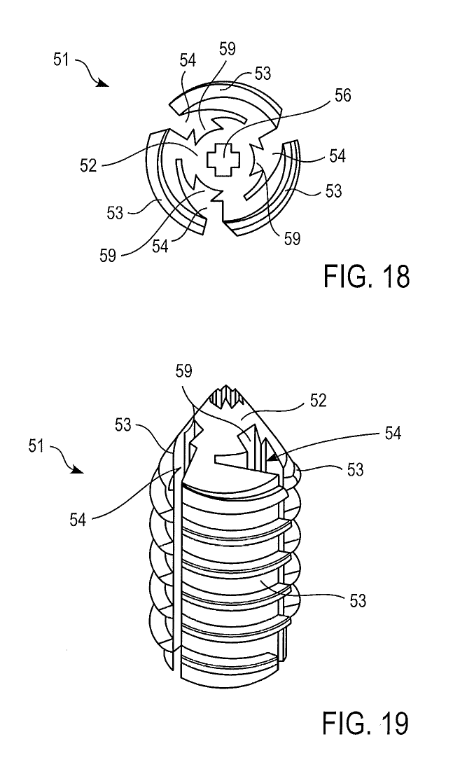

In an exemplary embodiment, FIG. 18 shows a view on the rear end of an exemplary embodiment of a fixation implant, which corresponds to a large extent to the fixation implant depicted in FIG. 13 based on the cross-sectional view. In FIG. 18, the exemplary fixation implant is given the overall reference number 51. Like the exemplary embodiment shown in FIG. 13, the exemplary fixation implant 51 of FIG. 18 has three wing-like shell segments 53 that are coupled radially movable to a body 52. Between the body 52 and the wing-like shell segments 53, in each case areas 54 are recessed that can be filled at least in places with an expandable material. In the depicted embodiment, for example, the entire area 54 is not back-filled with expandable material. Rather, grooves 59 that take up the expandable material are recessed in the areas 54. For reasons of better clarity, the depiction of the expandable material was eliminated. The expandable material can be an elastically compressible material and/or a material that can swell upon contact with bodily fluids. The non-expandable carrier material of the body 52 can include, for example, polylactide (PLA). On its rear end, the fixation implant 51 has a central receptacle 56 for an insertion instrument. The central receptacle 56 extends from a rear end area into the interior of the body 52. On the outer wall of the wing-like shell segments 53, threaded sections are designed, so that a ligament transplant can be better held, and the fixation implant in the bone canal can find a better anchoring.

In an exemplary embodiment, FIG. 19 shows a perspective view of the exemplary fixation implant 51 according to FIG. 18 with a view toward the front section when used in accordance with an exemplary aspect. The three shell-like segments that are coupled axially to the body 52 are in each case indicated at 53. The area between the shell-like segments 53 and the body 52 is provided with the reference number 54. For reasons of better clarity, a depiction of the expandable material was eliminated. The front section of the fixation implant 51 is designed to run together conically and has a kind of drill bit. The threaded sections molded on the outside of the shell-like segment 53 are clearly visible. The grooves provided in the areas 54 between the body 52 and the shell-like segments 53 for taking up the expandable material are indicated at 59. The shell-like segments 53 are coupled in an elastic manner to the body 52 of the fixation implant 51. As a result, the latter can be pressed together during insertion of the fixation implant 51. Because of the expandable material, for example because of a material that can swell upon contact with bodily fluids, the shell-like segments 53 with the threaded sections are pressed outward against the wall and the absorbing material. As expandable material, for example, a material that can swell upon contact with bodily fluid is used. Examples in this respect are swellable hydrogels, for example, co-polymers based on methyl methacrylate and N-vinyl pyrrolidone, available from, for example, Osmed GmbH, Hartheim, Germany.

The exemplary embodiments shown in FIGS. 1 to 19 can be employed, for example, using a material that can swell upon contact with a bodily fluid. In place of a swellable material, an elastically compressible material or a foamable material can also be used. Examples in this respect are polyurethane, polysiloxanes, polyolefins, soft polyvinyl chloride, synthetic rubber, thermoplastic elastomers, and other polymers, as described in “Elastomers for Biomedical Applications,” J Biomater Sci Polym Ed. 1998; 9(6): 561-626 or in “Encyclopedia of Biomaterials and Biomedical Engineering,” eds. Wnek, G. and Bowlin, G., Marcel Dekker, Inc., ISBN-10: 0824755561, the contents of which are incorporated by reference herein in their entireties. Combinations of elastically compressible and/or foamable and/or swellable materials can also be used.

For example, the materials that can be used for the fixation implant can be biocompatible and can be resorbable or else non-resorbable. Non-resorbable polymers can include biocompatible polymers, such as, e.g., polyethylene, polypropylene, polyethylene terephthalate, polyether ketone, polyether ether ketone, polyvinyl chloride, polycarbonate, polyamides, polyimides, polystyrene, polyacrylamide, polybutadiene, polytetrafluoroethylene, polyurethane, polysiloxane-elastomers, polyether ether ketone, polysulfone, polyether imides, polyacetates, poly-paraphenylene, terephthalamide, silicones, and carbon- or glass-fiber-reinforced composite materials. In an exemplary embodiment, hydrogels can also be of natural or synthetic origin, which swell by the absorption of water but do not dissolve, such as, e.g., poly-2-hydroxyethyl-methacrylate (PHEMA).

Resorbable or partially resorbable polymers which can be used can be polyhydroxy ester, polyorthoester, polyanhydride, polydioxanone, polyphosphazene, polyhydroxyalkanoate, polypropylene fumerate, polyester amide, polyethylene fumerate, polylactide, polyglycolide, poly-ε-caprolactone (PCL), polytrimethylene carbonate, polyphosphazene, polyphosphates, polyvinyl alcohol, polymaleic acid (b) or polymaleic acid ester, poly-p-dioxanone and copolymers, modifications or mixtures of the same. As examples, lactate/glycolide copolymers, lactate/tetramethylene glycolide copolymers, lactate-trimethylene carbonate copolymers, lactate/alpha-valerolactone copolymers, lactate/ε-caprolactone copolymers, polydepsipeptides (glycine-DL-lactate copolymer or lactate/ethylene oxide copolymers), or, from the group of polyhydroxyalkanoates, e.g., PHB [polyhydroxybutyrate)], or PHB/PHV (polyhydroxybutyrate/-valerate), can be mentioned.

Also suitable are, for example, mixtures or copolymers with vinyl polymers, e.g., based on poly-β-maleic acid, aliphatic polyamides, aliphatic polyurethanes, e.g., polyurethanes that consist of polyethylene glycol (PEG) diols or polycaprolactone diols and diisocyanates, polypeptides, e.g., synthetic polyamino acids and poly-α-amino acids, e.g., poly-β-lysine or polybenzyl glutamate, polyurethane-diol glycosaminoglycan, polysaccharides, e.g., dextran derivatives, chitin or chitosan derivatives or hyaluronic acid esters, alginates, gelatins or cellulose derivatives, modified proteins, e.g., partially cross-linked collagen or fibrin, or modified carbohydrate polymers.

For example, to match the elasticity or the swelling behavior, polymers can be mixed with softeners, e.g., from monomers or oligomers of the same polymers, from biocompatible softeners, such as, e.g., acetyl tributyl citrate, citric acid, etc.

For example, so-called super-absorbers, which are able to take up a multiple of their own weight, for example, up to 1000 times, of liquids, for example, water or distilled water, can also be used. Chemically, in the current state of the art, the super-absorber is a copolymer that consists of acrylic acid (propenoic acid, C₃H₄O₂) and sodium acrylate (sodium salt of acrylic acid, NaC₃H₃O₂).

For example, to increase the swellability of materials or only to make the swellability possible, hydrophilic substances, for example, in the form of particles or nanoparticles, can be added to the polymers. These particles produce an osmotic effect. Exemplary substances can include salts, such as, e.g., sodium chloride, but also calcium phosphates, such as, e.g., monocalcium phosphate monohydrate, monocalcium phosphate anhydrate, dicalcium phosphate dihydrate, dicalcium phosphate anhydrate, tetracalcium phosphate, calcium orthophosphate, calcium pyrophosphate, α-tricalcium phosphate, 13-tricalcium phosphate, apatites, such as, e.g., hydroxyl apatite, calcium sulfates, sodium sulfates, sodium phosphates, etc.

An exemplary feature of exemplary embodiments is that they can increase their external diameter immediately after their insertion into a receptacle hole in the bone, i.e., ab initio, and thus exert pressure on the surrounding bone substance. The selection of the materials can be carried out in this case in such a way that an expansion pressure of 5 MPa is not exceeded. For example, the fixation implant is designed in such a way that it has a low elastic compressibility. As a result, on the one hand, the insertion of the fixation implant into the bone hole can be facilitated, and, on the other hand, an anchoring in the bone hole and a clamp fixing of a ligament transplant ab initio can be ensured independently of swelling or an increase in diameter owing to liquid absorption and retention.

It will be appreciated by those skilled in the art that the present invention can be embodied in other specific forms without departing from the spirit or essential characteristics thereof. The presently disclosed embodiments are therefore considered in all respects to be illustrative and not restricted. The scope of the invention is indicated by the appended claims rather than the foregoing description and all changes that come within the meaning and range and equivalence thereof are intended to be embraced therein.

What is claimed is:

1. A fixation implant for tightly securing a ligament transplant on or in a bone, the fixation implant comprising:
an elongated body that includes a front end, and at least partially contains an expandable material that, after implantation, exerts an expansion pressure on a surrounding bone substance,
wherein the elongated body includes an outer wall which includes, at some areas, profiles that extend substantially transverse to a longitudinal direction,
wherein the elongated body on its rear end has engagement means for an insertion instrument, or an opening in a central receptacle for an expansion body,
wherein the expandable material is arranged substantially along the entire longitudinal extent of the elongated body, and
wherein the expandable material is arranged such that, upon expansion, the expandable material exerts pressure on the areas that includes the profiles in such a way that the external diameter of the body increases.

2. The fixation implant according to claim 1, wherein the elongated body is a solid body without a central through-hole, wherein the elongated body consists entirely of the expandable material, and wherein the engagement means is arranged in a cover plate that is applied on the rear end of the elongated body and includes a non-expandable material, which is connected permanently to the elongated body.

3. The fixation implant according to claim 1, wherein the body is a solid body without a central through-hole, wherein the elongated body comprises a composite structure that includes the expandable material and a non-expandable material forming an outer surface of the body, and wherein the fixation implant is structured such that the external diameter of the elongated body increases when an expansion pressure is exerted.

4. The fixation implant according to claim 3, further comprising structure defining at least one slot that runs axially and radially into the body.

5. The fixation implant according to claim 1, wherein the elongated body includes a non-expandable material and has an expansion area that is defined by axial slots, and the central receptacle is a receptacle, conically tapered to the front end, for engaging an expansion cone that includes the expandable material.

6. The fixation implant according to claim 5, wherein the expansion cone on its rear side facing the front end has a cover plate that includes a non-expandable material.

7. The fixation implant according to claim 5, wherein the conical receptacle is equipped with a ribbing that engages a structure that is provided on the outer wall of the expansion cone.

8. The fixation implant according to claim 1, wherein the elongated body comprises one or more shell segments each including a non-expandable material, which surround the expandable material and can move radially outward during a radial expansion resulting in an enlargement of the external diameter of the elongated body.

9. The fixation implant according to claim 8, wherein the shell segments are equipped on their rear ends with recesses for free ends of ligament transplants, wherein the recesses are open toward the outer surface of the elongated body.

10. The fixation implant according to claim 9, wherein the recesses are conically tapered from an insertion opening in the direction of the front end.

11. The fixation implant according to claim 1, wherein the body has a central structural skeleton that includes a non-expandable material, which has receptacles filled with the expandable material that run in the peripheral direction or axially and that are open toward the periphery of the body, and wherein the engagement means is arranged in or on the central structural skeleton.

12. The fixation implant according to claim 11, wherein the expandable material is covered in each case by a layer of non-expandable material toward the periphery of the body.

13. The fixation implant according to claim 12, wherein the profiles are provided at least partially on the layer of non-expandable material.

14. The fixation implant according to claim 1, wherein the body includes a non-expandable material and contains structure defining canals, furrows, and/or receptacles that open toward the periphery and that are filled with the expandable material.

15. The fixation implant according to claim 1, wherein the body comprises a structural skeleton that includes a non-expandable material, which is provided with radially deformable areas that radially contact areas filled with expandable material.

16. The fixation implant according to claim 15, wherein the radially deformable areas include shell segments and are coupled axially on one side on the periphery of the body.

17. The fixation implant according to claim 1, wherein the expandable material comprises an elastically compressible plastic material.

18. The fixation implant according to claim 17, wherein the expandable material comprises a material selected from the group consisting of a polyurethane, polyester urethane, polysiloxane, polyvinyl chloride, poly-ε-caprolactone, trimethylene carbonate, softened polymer, and a combination thereof.

19. The fixation implant according to claim 1, wherein the expandable material comprises a material that can swell upon contact with a bodily fluid.

20. The fixation implant according to claim 19, wherein the material that can swell upon contact with a bodily fluid is a swellable hydrogel.

21. The fixation implant according to claim 19, wherein the elongated body contains structure defining holes, canals and/or capillaries for the transport of bodily fluid to the expanding material.

22. The fixation implant according to claim 19, wherein the material that can swell upon contact with a bodily fluid is mixed with a substance that has an osmotic action.

23. The fixation implant according to claim 21, wherein the substance that has an osmotic action comprise a salt.

24. The fixation implant according to claim 19, wherein the body comprises a resorbable plastic material in at least some areas, and said resorbable plastic material has a molecular weight of less than 100,000 g/mol.

25. The fixation implant according to claim 19, wherein in at least some areas, the elongated body includes a material selected from the group consisting of a non-resorbable swellable material, a resorbable swellable material and a combination thereof.

26. The fixation implant according to claim 25, wherein the elongated body comprises at least one polymer selected from the group consisting of a homopolymer, copolymer or oligomer of a polyhydroxy acid, polyester, polyorthoester, polyanhydride, polydioxanone, polydioxanonedione, polyester amide, polyamino acid, polyamide, or polycarbonate, polycaprolactone, polylactide, polyglycolide, tyrosine-reacted polycarbonate, polyanhydride, polyorthoester, polyphosphazene, polyethylene, polyester, polyvinyl alcohol, polyacrylonitrile, polyamide, poly-2-hydroxyethyl methacrylate, polysiloxane, polytetrafluoroethylene, poly-paraphenylene, terephthalamide, polyacryl ether ketone, polyether ketone, polyether ether ketone, cellulose, carbon-fiber-reinforced or glass-fiber-reinforced composite material, and a combination thereof.

27. The fixation implant according to claim 1, wherein the elongated body comprises a non-swellable material selected from the group consisting of a resorbable polymer, bioceramic, polyether ether ketone, polyester imide, polyethylene, and a combination thereof.

28. The fixation implant according to claim 1, comprising a material that is resorbable in a human body.

29. The fixation implant according to claim 1, wherein the elongated body is an interference screw.

30. The fixation implant according to claim 1, wherein the fixation implant is produced in an injection-molding process.

31. The fixation implant according to claim 19, wherein the material that can swell upon contact with a bodily fluid is a co-polymer based on methyl methacrylate and N-vinyl pyrrolidone.

32. A method of tightly securing a ligament transplant in or on a bone, the method comprising:
implanting the fixation implant according to claim 1 into a surrounding bone substance.

33. The method according to claim 31, further comprising introducing the expandable material to conditions which cause the expandable material to expand so as to exert expansion pressure on the surrounding bone substance.
